(19) United States
(12) Patent Application Publication
(10) Pub. No.: US 2006/0293691 A1
(43) Pub. Date: Dec. 28, 2006

(54) Apparatus and methods for performing cerclage

(76) Inventors: Avir Mitra, Voorhees, NJ (US); Amitabha Mitra, Voorhees, NJ (US)

Correspondence Address: FLASTER/GREENBERG P.C.;8 PENN CENTER, 1628 JOHN F. KENNEDY BLVD., 15TH FLOOR, PHILADELPHIA, PA, 19103, US

(21) Appl. No.: 11/446,940

(22) Filed: Jun. 5, 2006

Related U.S. Application Data
(60) Provisional application No. 60/687,961, filed on Jun. 7, 2005.

(51) Int. Cl. A61B 17/58

(52) U.S. Cl. 606/103

(57) ABSTRACT

Disclosed are apparatus and methods for performing cerclage. In some aspects of the present invention, the apparatus is designed to both compress a fracture and aid the operator in applying cerclage thereto. Cerclage is facilitated by providing a passageway internal to the apparatus that encircles the fracture during compression of the fracture with the apparatus. The internal passageway allows a cerclage material to be threaded therethrough without disruption or damage to surrounding tissue, skin, or the like. Upon removal of the apparatus, cerclage may be performed by tightening or otherwise tying off the cerclage material. In some embodiments of the present invention, the apparatus portions comprising the internal passageway are removable to allow such passageways to be discarded after each use, thereby simplifying sterilization and the like thereof. In other aspects of the present invention, interlocking components are provided to facilitate threading of the cerclage material through the apparatus.

### CROSS-REFERENCE TO RELATED APPLICATIONS

This application claims the benefit of the U.S. provisional patent application entitled “Cerclage Clamp”, having Ser. No. 60/687,961, filed Jun. 7, 2005, which is incorporated by reference in its entirety as if fully set forth herein.

### BACKGROUND OF THE INVENTION

Embodiments of the present invention generally relate to apparatus and methods for efficiently and effectively performing cerclage. More specifically, the present invention relates to apparatus and methods for applying cerclage to bone fractures during bone reparation and/or fixation surgery.

Cerclage is the use of an encircling loop or ring to bring together the portions of detached, splintered, shattered, and/or fractured body parts such as detached muscles and/or tendons, fractured or splintered bones, bone fragments, broken patellas, and the like. In some cerclage applications, the encircling loop or ring may be made of wire, metal, plastic, and/or any future biodegradable composition, and such materials may have any one of a variety of compositions and gauges. Cerclage has been shown to be a useful technique for the reduction of fractures of the long bones of the body. It is particularly useful for reduction of fractures such as spiral fractures and other splitting fractures, as well as reparation, fixations, and the like in which hardware (e.g., plates, screws, etc.) is not feasible or sufficient to hold the fractured body parts together as they heal. However, cerclage materials (e.g., cerclage wire, dental wire, suture thread, etc.) can be difficult to apply.

Several methods are known for reducing and fixating bone fractures via cerclage. One such method includes drawing the fragments of a bone fracture together, wrapping tensioned cables or wires around the fragments, and crimping, clamping, tying-off, or otherwise securing the tensioned cable or wire in place to retain the position of the fragments as they heal. To perform cerclage, a surgeon typically visually assesses the best route for passing the tensioned cable or wire around the fracture site and uses a standard wire passer, a curved needle, or the like to thread the cerclage cable or wire therethrough. Or, in even simpler embodiments, the surgeon simply uses his or her hand to guide the cerclage cables or wires. However, such methods are sometimes difficult to perform as the insertion and application of such bone reduction cables and wires requires care to avoid cutting off the blood supply to the fracture site and piercing or other disruption of the soft tissues (e.g., arteries and nerves) that surround the fracture site. Additionally, such methods often require several and/or large incisions to ensure that the surgeon has complete access to, and visualization of, the entire circumference of the fracture site.

Similarly, several apparatus are known for facilitating the performance of cerclage. One such apparatus is a cerclage wire passer that includes a handle at a first end and an ‘S-shaped’ segment at a second end. The end of the S-shaped segment opposite the attached handle includes an eyelet hole through which a cerclage wire may be hooked. During use, a surgeon inserts the S-shaped segment into the bone fracture area and rotates it under and around the fracture site such that the eyelet hole is exposed on the opposite side of the fracture site from which the apparatus is inserted. The surgeon then inserts a wire through the eyelet hole and hooks the wire such that it will remain within the eyelet hole during removal of the wire passer. As the surgeon removes the cerclage wire passer from the fracture site, the wire is pulled under and around the fracture site in the opposite manner to which the apparatus was originally inserted. Thereafter, the wire may be cut and its two ends may be tied or otherwise secured to hold the fracture segments in place. During use of such apparatus, care must be taken to ensure that soft tissue, which typically surrounds such fracture sites, is not damaged or captured by the wire. Additional devices and techniques may be required to reduce, compress, or retain fractures prior to application of the cerclage wire. As additional devices and techniques are required, additional time and personnel may also be required. Or, the surgeon may be required to use an additional hand for the manipulation of the instruments, thereby increasing the complexity of the procedure.

Another similar apparatus for performing cerclage includes a pair of passer members (i.e., devices similar to the aforementioned cerclage wire passers that aid in wrapping the cerclage wire around the fracture site), each having curved cannulated ends designed to encircle the fracture site when the ends of the passer members are mated together. The cannulated ends allow a wire such as a cerclage wire to be passed therethrough, thereby encircling the fracture site. In one implementation, the passer members each include magnetic tips having opposite polarity such that the tips are magnetically drawn together. In other implementations, magnets are included in the handles of the passer members to aid mating of the members and retaining such members in a mated position (e.g., around the fracture site). In yet another implementation of such apparatus, the wire has a magnetic tip that allows a passer member having a magnetic tip of opposite polarity to draw the wire through a cannulated end and/or around the fracture site.

Other cerclage devices include the capability to drill through bone to apply cerclage through, rather than around, a bone fracture. Some such devices are scissor-like devices having arcuate cannulated sections attached to the ends thereof that serve as drill and/or wire guides. The arcuate shape of the cannulated sections allow the sections to be inserted into converging angular holes drilled through the cortex of the bone on each side of the fracture. When the cannulated arcuate sections are interlocked within the medula of the bone, a continuous passageway is formed that allows a wire or the like to be fed therethrough. Additionally, the arcuate shape of the cannulated sections allow a flexible cable with a drilling burr to be passed through a first cannulated section to facilitate drilling of the aforementioned converging angular holes. In such scenarios, the second cannulated section may be equipped with a tapered pin adapted to be received into a recess in the leading edge of the burr upon completion of the drilling through the bone.

Furthermore, some cerclage apparatus include a housing having a handgrip at a first proximal end and a tapered tip with an opening therein at a second distal end. Within the housing is an extendable tongue formed from a material such as spring metal that curls when the tongue is extended outside of its housing. Such tongue also has an eyelet at its distal end. During use of the apparatus, the surgeon extends the tongue outside its housing via a plunger such that the tongue encircles the bone. Once the distal end of the tongue emerges on the opposing side of the fracture site, the surgeon inserts and attaches a cerclage wire into the tongue's eyelet. The tongue is then retracted, thereby pulling the cerclage wire around the bone. Use of such apparatus requires two passages of the tongue around the bone. The first passage places the tongue around the bone, and the second passage pulls the cerclage wire around the bone as the tongue is retracted.

### BRIEF SUMMARY OF THE INVENTION

Briefly stated, in one aspect of the present invention, an apparatus for applying cerclage to a plurality of segments is provided. This apparatus includes two jaw members, each of the jaw members including at least one cannulated end, and each of the cannulated ends including at least two apertures, and at least one of the apertures is located at each terminus of the cannulated end; two handle members for opening and closing the jaw members relative to each other; and at least one hinge for coupling the jaws to each other and for coupling the jaw members to the handle members; wherein the jaw members are configured relative to each other to draw the plurality of segments together to a desired position as an operator manipulates the jaw members from an open position to a closed position; and wherein the cannulated ends are configured relative to the jaw members to engage each other when the apparatus is manipulated to a closed position; and wherein the engagement mates cannulated portions of each of the cannulated ends together to form one continuous passageway through which a cerclage material may be threaded.

In another aspect of the present invention, a method for applying cerclage to a plurality of segments is provided. This method includes providing a cerclage implement having jaw members, the jaw members pivotally coupled to each other, and each of the jaw members including at least one cannulated end; surrounding the plurality of the segments with the jaw members; drawing the plurality of the segments together to a desired position via pivoting of the jaw members while simultaneously engaging the cannulated ends of the jaw members, the engagement mating cannulated portions of each of the cannulated ends together to form one continuous passageway through which a cerclage material may be threaded; threading at least one cerclage material through the continuous passageway; removing the cerclage implement from the plurality of segments; and securing the cerclage material.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments that are presently preferred. It should be understood, however, that the invention is not limited to the precise arrangements and instrumentalities shown. In the drawings:

FIG. 1 is a perspective view of an apparatus for performing cerclage in accordance with an embodiment of the present invention, wherein the apparatus is depicted in an open position;

FIG. 2A is a perspective view of an apparatus for performing cerclage in accordance with an embodiment of the present invention, wherein the apparatus is depicted in a closed position;

FIG. 2B is a perspective view of an apparatus for performing cerclage in accordance with an embodiment of the present invention, wherein the apparatus is depicted in a closed position with a cerclage material threaded through its jaws;

FIG. 3A is a cross-sectional view of the apparatus depicted in FIG. 1 taken along lines 3A-3A of FIG. 1;

FIG. 3B is a cross-sectional view of the apparatus depicted in FIG. 1 taken along lines 3B-3B of FIG. 3A;

FIG. 4 is a right side view of the apparatus depicted in FIG. 1 in accordance with an embodiment of the present invention;

FIG. 5A is a magnified view of an alternate embodiment of cannulated members having cannulated tips with interlocking mating faces for inclusion in an apparatus created in accordance with the present invention;

FIG. 5B is a cross-sectional view of the cannulated members having cannulated tips with interlocking mating faces depicted in FIG. 5A taken along lines 5B-5B of FIG. 1;

FIG. 6A is a perspective view of an apparatus having detachable tips in accordance with one embodiment of the present invention;

FIG. 6B is a cross-sectional view of the cannulated members and cannulated tips of the detachable ends taken along lines 6B-6B of FIG. 6A;

FIG. 6C is an exploded view of the apparatus depicted in FIG. 6A in accordance with one embodiment of the present invention;

FIG. 7 is a flowchart of an exemplary method for performing cerclage in accordance with one embodiment of the present invention;

FIG. 8 illustrates the step of encircling an exemplary metacarpal bone with the jaws of an implement in accordance with one method of the present invention.

FIG. 9A illustrates the step of threading a first end of a cerclage material into an implement in accordance with one method of the present invention;

FIG. 9B illustrates an implement through which a cerclage material has been fully threaded in accordance with one method of present invention; and

FIG. 10 illustrates the step of removing an implement from a facture site in accordance with one method of the present invention.

### DETAILED DESCRIPTION OF THE INVENTION

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a fragment” includes a plurality of fragments. Thus, for example, a reference to “a method” includes one or more methods, and/or steps of the type described herein and/or which will become apparent to those persons skilled in the art upon reading this disclosure.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, constructs and materials are now described. All publications mentioned herein are incorporated herein by reference in their entirety. Where there are discrepancies in terms and definitions used in references that are incorporated by reference, the terms used in this application shall have the definitions given herein.

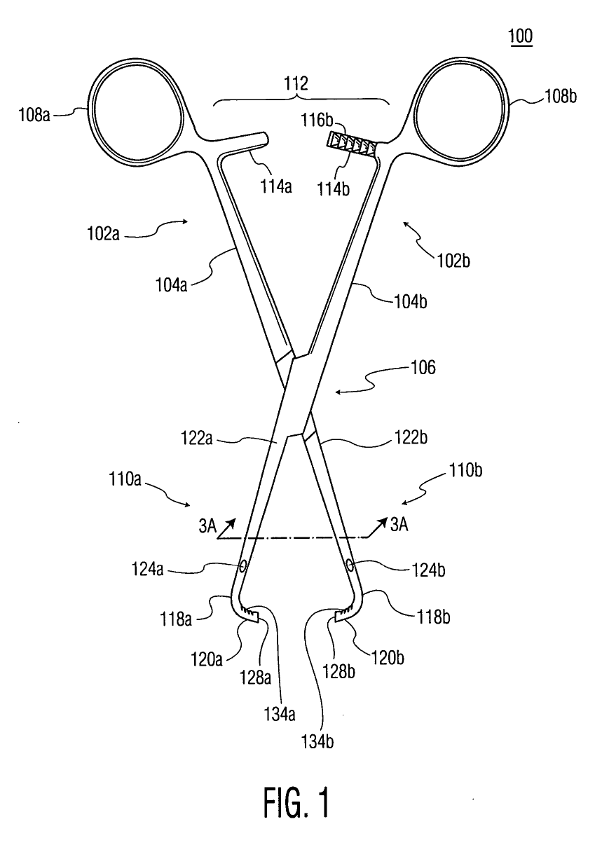

Referring first to FIG. 1, depicted is a perspective view of an apparatus for performing cerclage in accordance with one embodiment of the present invention. In the depicted embodiment of the present invention, implement 100 includes, inter alia, handles 102, hinge 106, and jaws 110.

In some embodiments of the present invention, handles 102a and 102b include handle members 104a and 104b, respectively. Handle members 104a and 104b pivot with respect to each other via hinge 106 to allow an implement operator to vary the distance of cannulated ends 118, and consequently cannulated tips 120, from each other. The ability to vary such distance allows a surgeon or implement operator to manipulate implement 100 as necessary to place jaw members 122 around a bone fracture site, a muscle separation site, a broken patella, etc. Although the apparatus and methods of the present invention will be discussed herein with respect to applying cerclage to a bone fracture, such application is exemplary and it should be understood that the present invention is not so limited and it may be used for other non-bone fracture applications.

In some aspects of the present invention, one or more digit inserts 108 may be coupled to the distal ends of handle members 104. For example, as depicted in FIG. 1, digit inserts may be in the form of opposing loops attached to the outwardly facing sides of the distal ends of handle members 104. Digit inserts 108 allow an implement operator to insert his digits (e.g., finger, thumb, etc.) therethrough such that a better grip of implement 100 is achieved. Furthermore, a more secure grip minimizes the potential of dropping implement 100 during use in surgery or the like. Although FIG. 1 depicts digit inserts 108 as loops, other forms of digit inserts or other gripping mechanisms (e.g., handgrips) may be substituted without departing from the scope of the present invention.

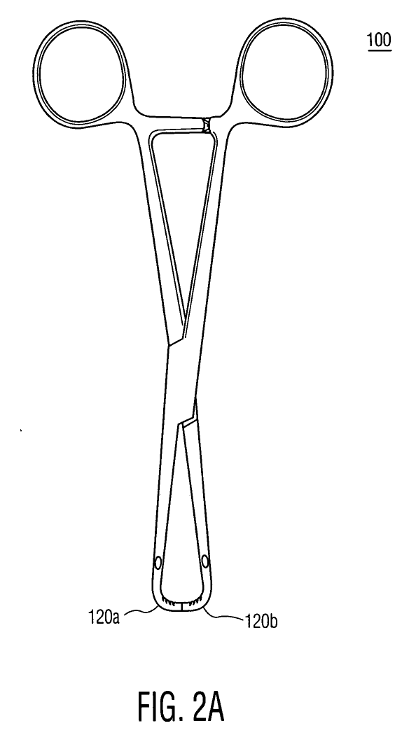

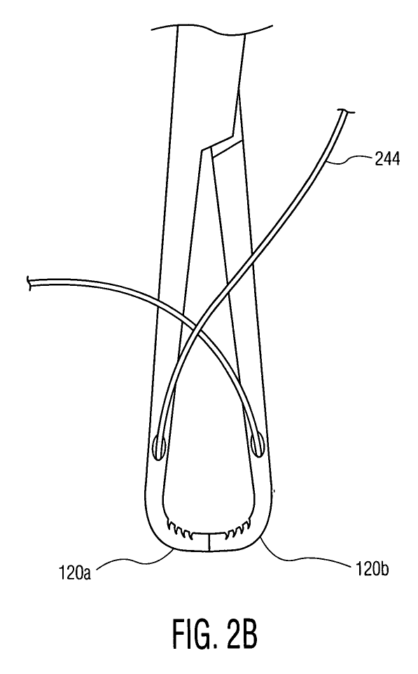

In another aspect of the present invention, one or more locking mechanisms 112 may also be coupled to one or more distal ends of handle members 104. For example, as depicted in FIG. 1, locking mechanism 112 includes toothed protrusions 114 extending inwardly from the inwardly facing surfaces of each of handle members 104. An implement operator may interlock teeth 116 by squeezing handle members towards each other until teeth 116a (not shown), which are located on the back side of toothed protrusion 114a in the same manner as teeth 116b are located upon toothed protrusion 114b, overlap with teeth 116b or vice versa. Thereafter, the configuration of handles 102 and toothed protrusions 114 relative to each other as well as the inwardly directed pressure exerted on handle members 104 by hinge 106 hold implement 100 in a locked position. Such locked position holds jaw members 122, and consequently cannulated ends 118 and cannulated tips 120, in a fixed position. For example, such fixed position may include a closed position, as depicted in FIG. 2A, in which cannulated tips 120 are mated firmly together such that a cerclage material such as cerclage material 244 may be more easily passed therethrough as depicted in FIG. 2B. Locking mechanism may thereafter be moved to an unlocked position by exerting opposing lateral pressure on each of handles 102 such that teeth 116a (not shown) may clear teeth 116b, or vice versa, as handles 102 are opened away from each other until toothed protrusions 116 no longer overlap. The size and number of interlocking teeth 116 may be adjusted to accommodate various applications. For example, only two interlocking teeth 116 may be required for a two-position (e.g., open-closed) applications. Although FIG. 1 depicts locking mechanism 112 as including interlocking teeth 116, other locking mechanisms (e.g., pin-and-hole, latched mechanisms, magnetic locking mechanisms, etc.) may be substituted without departing from the scope of the present invention.

In some embodiments of the present invention, hinge 106 is included to facilitate rotation of handle members 104 relative to each other, thereby facilitating opening and closing of cannulated ends 118 and cannulated tips 120. For example, hinge 106 may be a single pin connected on either end to a first handle member and passing through the second handle member as is known in the art. Alternatively, one handle of implement 100 may be designed to fit within the opposing handle at hinge 106, thereby providing additional lateral support. However other hinges may be substituted without departing from the scope of the present invention.

In the embodiment of the present invention depicted in FIG. 1, jaws 110 include, inter alia, jaw members 122, cannulated ends 118, and cannulated tips 120. In one aspect of the present invention, jaw members 122 are formed as extensions of handles 102, which extend from handles 102 beyond hinge 106. In one use of the present invention, during surgery, jaw members 122 are placed around bone fracture fragments and are closed via manipulation of handles 102. When jaw members 122 are closed around the fractured bone and/or fractured bone fragments, jaw members 122 facilitate operative reduction by drawing the fracture fragments together circumferentially. Additionally, jaw members 122, when open, can easily be manipulated around the fractured bone and/or fractured bone fragments, thereby reducing the risk of damage to surrounding soft tissues. Furthermore, jaw members 122 are able to encircle the fracture site with a lesser quantity of incisions, smaller incisions, and/or incisions made on only one side of the fracture site since complete visualization of the entire circumference of the fracture site is not always required during implementation of the systems and methods of the present invention.

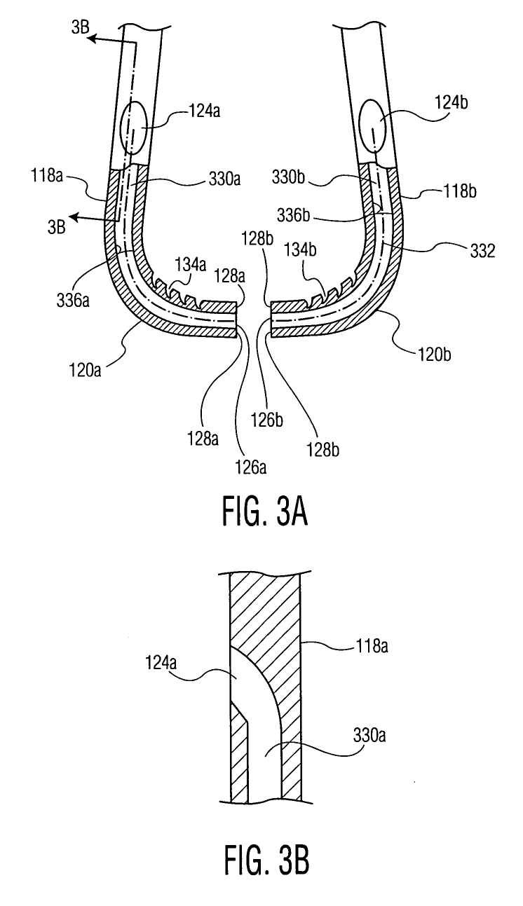

Referring now to FIG. 3A, each jaw member 122 (FIG. 1) includes a cannulated end 118 at its distal end. Each cannulated end 118 includes, inter alia, aperture 124, cannulated tip 120, and passageway 330. Each aperture 124 extends from its respective passageway 330 through an exterior surface of its respective cannulated end 118 such that aperture 124 is perpendicular to the longitudinal axis of its respective passageway 330.

When implement 100 is in the closed position as depicted in FIGS. 2A and 2B such that jaws 110 (FIG. 1) are closed, passageways 330a and 330b combine to create a single passageway 332. Passageway 332 extends from a first aperture 124 through its respective passageway 330 through its respective mating face aperture 126 into the opposing mating face aperture 126 through the opposing passageway 330 and terminating at the opposing aperture 124. Passageway 332 is formed when mating faces 128a and 128b of cannulated tips 120a and 120b, respectively, as also depicted in FIG. 1, interlock such that passageways 330a and 330b form a continuous channel for the passage of a cerclage material therethrough. Such mating or interlocking of cannulated tips 120 may include butting or otherwise interconnecting mating faces 128 in virtually any manner in which the joint formed between cannulated tips 120 does not create an obstruction to the passage of a cerclage material therethrough. The cerclage material may then be inserted into either of apertures 124a or 124b, whereupon it passes first through passageway 330a or 330b, respectively, second through passageway 330b or 330a, respectively, and thereafter exits at the remaining aperture 124b or 124a, respectively. FIG. 2C depicts the resulting implement 100 having a cerclage material threaded therethrough. In one embodiment of the present invention, both apertures 124a and 124b are located at the respective terminus of passageway 332, thereby facilitating threading of the cerclage material and forcing the cerclage material to exit passageway 332 at the aperture opposite the aperture into which the cerclage material is first inserted.

In some aspects of the present invention, apertures 124, mating face apertures 126, passageways 330, and passageways 332 are sized and/or configured to accommodate smooth passage therethrough of the cerclage material. For example, the aforementioned elements of implement 100 may be sized and/or configured to accommodate 24- or 26-gauge stainless steel cerclage wire or the like. Additionally, to further facilitate passage of the cerclage material, some embodiments of the present invention include inwardly facing surfaces 336 of passageways 330 that are smooth, polished, or are otherwise configured to minimize friction with or snagging of the cerclage material while it is being threaded through passageways 330 and/or 332 as the tip of the cerclage material will generally slide along the inwardly facing surfaces 336 as the cerclage material is threaded therethrough. For example, in one aspect of the present invention, inwardly facing surfaces 336 of passageways 330 are configured without any sharp angles (e.g., such as the passageway depicted in FIG. 3B) to minimize snagging or other obstruction of the cerclage material.

Referring to FIG. 3B, depicted is one embodiment of the terminus of passageway 330a of cannulated end 118a in accordance with the present invention. As shown, as passageway 330 approaches aperture 124a it slopes toward aperture 124 such that an end of a cerclage material passed therethrough is guided to exit aperture 124. However, alternate embodiments of passageways 330 may be substituted without departing from the scope of the present invention.

Also, as depicted in FIGS. 1 and 3A, the inwardly facing exterior surfaces of cannulated ends 118 and/or cannulated tips 120 may optionally include serrations 134. In some aspects of the present invention, jaws 110 are sized such that when they are in a closed position, the inwardly facing exterior surfaces of cannulated ends 118 and/or cannulated tips 120, and therefore, serrations 134 if any, physically contact the encircled bone fracture fragments. These serrations provide additional friction during such physical contact, thereby increasing the likelihood that jaws 110 will maintain contact with the bone fracture fragments and, consequently, draw such bone fracture fragments into the position desired by the implement operator. That is, without the additional friction introduced by serrations 134, the likelihood of -slippage of inwardly facing exterior surfaces of cannulated ends 118 and/or cannulated tips 120 from the bone fracture fragments is increased, potentially resulting in multiple attempts by the implement operator to properly position the bone fracture fragments such that cerclage may be applied thereto. However, serrations 134 or the like are not required to implement the present invention. In fact, in some embodiments such as, but not limited to, muscle repair, serrations may be purposely omitted to prevent damage to the body part that will contact implement 100.

Referring back to FIG. 1, in some aspects of the present invention, implement 100 is manufactured from surgical steel, stainless steel, other metals or alloys as are known in the art. However, other materials (e.g., polycarbonate) may be substituted without departing from the scope hereof. The rigid nature of such materials allows implement 100 to forcefully engage fracture fragments and exert a compressive force across the fracture site. Such compression aids bone reduction and fixation.

For ease of manufacturing, implement 100 may initially be manufactured as two pieces and assembled via the addition of a pin or the like at hinge 106. That is, digit insert 108a, toothed protrusion 114a, handle member 104a, jaw member 122a, cannulated end 118a, and cannulated tip 120a would be manufactured as a single piece. Similarly, digit insert 108b, toothed protrusion 114b, handle member 104b, jaw member 122b, cannulated end 118b, and cannulated tip 120b would be manufactured as a single piece. The two pieces may then be assembled to form implement 100 with the addition of a pin as is known in the art. However, other methods of manufacturing and/or assembly may be used without departing from the scope hereof.

The actual size of the implement will vary depending on the size of the bone or fracture site upon which it will be used. For example, implements having a larger distance or other area between its jaws such as jaws 110 (as measured when the jaws are in a closed position) might be required to clamp a fractured tibia as opposed to a fractured phalange in a human finger. Similarly, a surgeon may require an implement having a larger distance or other area between its jaws to repair an adult bone than that required to repair the same child bone. For these and other reasons, a surgeon or other implement operator may require a set of implements 100 to accommodate a variety of applications and/or operations, wherein each implement is sized to the specific size and diameter of the bone to which it will be applied.

In another aspect of the present invention, each implement is provided with one or more identification devices (e.g., a molded piece for mounting upon an implement) to identify information such as the size of the distance or other area between its jaws, the bones for which the implement is sized, the patients (e.g., adult, child, etc.) for which the implement is sized, whether the implement is intended for right-handed or left-handed use, etc. However, such identification devices are optional and are not required to achieve the goals of the present invention.

In some embodiments of the present invention, cannulated members and/or cannulated tips are mounted to the distal ends of a surgical clamp, surgical clasp, or the like as known in the art. Appending such existing devices minimizes the learning curve associated with the present invention since the method of operation of the surgical clamp, surgical clasp, or the like is widely known in the art. Similarly, experienced surgeons are likely to have acquired skill in using or manipulating the surgical clamp, surgical clasp, or the like, which skill may be carried over for use of an implement in the form of a modified surgical clamp, surgical clasp, or the like in accordance with the present invention.

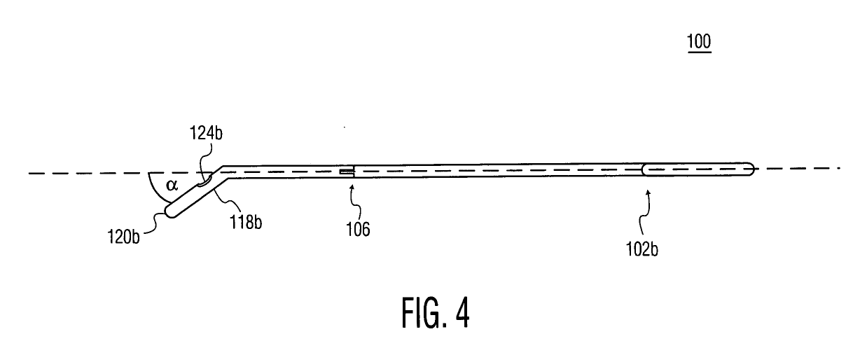

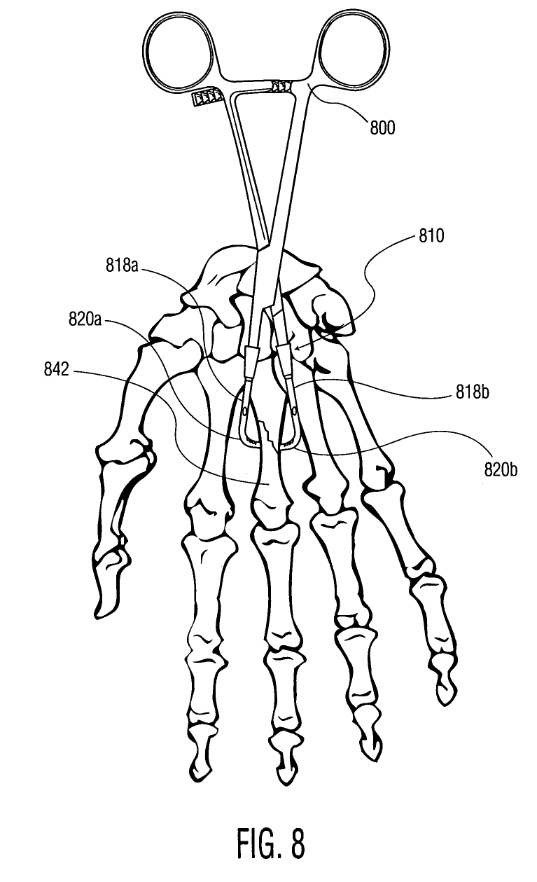

Turning now to FIG. 4, depicted is a right side view of implement 100 including handle 102b, hinge 106, jaw 110b, aperture 124b, cannulated end 118b, and cannulated tip 120b as discussed in greater detail above with respect to FIG. 1. FIG. 4 illustrates the downwardly angled configuration of cannulated end 118b and cannulated tip 120b relative to the longitudinal axis of hinge 106 and handles 102. In the depicted embodiment, angle α is approximately forty-five degrees. Such an angle facilitates encircling cannulated ends and tips 118 and 120, respectively, around a fractured bone such as the exemplary metacarpal bone depicted in FIG. 8, since the implement operator's angle of approach for implement 100 during application of cerclage to such types of fractured bones tends to be approximately forty-five to ninety degrees relative to the longitudinal axis of the metacarpal bone.

Furthermore, the angle α depicted in FIG. 4 provides the implement operator with a clearer view of the operative field as compared to an implement having a lesser angle α, such as zero. This occurs because the implement operator is not required to hold the implement at a ninety degree angle relative to the fractured bone to completely encircle it, which would thereby result in an obstructed view. Rather, an angle a of approximately forty five degrees allows implement 100 to be held at an angle of approximately forty five degrees relative to the fractured bone while completely encircling it and providing a clear visual path for the implement operator. Although implement 100 is depicted in FIG. 4 with an angle α of approximately 45 degrees, angle a may be any suitable angle from zero to approximately ninety degrees and may vary with criteria such as the method of use of the specific implement, the intended application of the specific implement (e.g., type of fracture to which cerclage will be applied), etc.

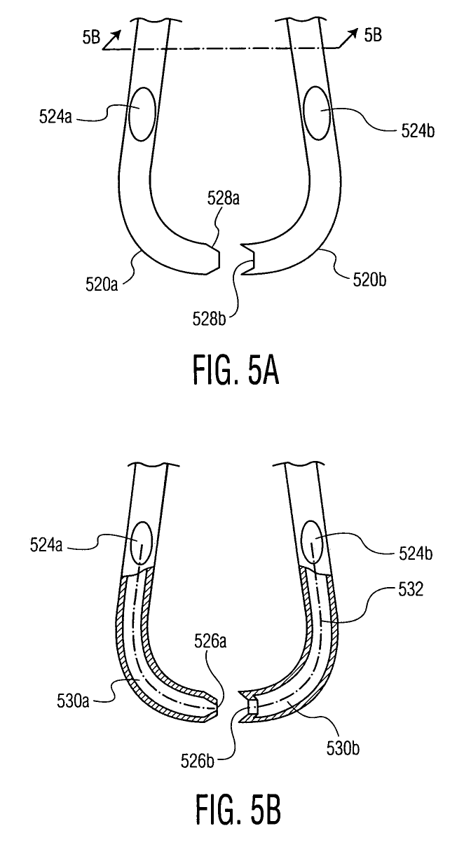

Referring now to FIG. 5, depicted is an alternate embodiment of cannulated tips 120 as depicted in FIG. 1. Cannulated tips 520 have interlocking mating faces in accordance with an embodiment of the present invention. As depicted, a first cannulated tip 520a includes a protruding mating face 528a and a second cannulated tip 520b includes a receiving mating face 528b. When an implement such as implement 100 to which such cannulated tips 520 are attached is closed such that cannulated tips 528 mate, protruding mating face 528a is inserted into receiving mating face 528b such that passageways 530a (FIG. 5B) and 530b (FIG. 5B) form one continuous passageway 532 (FIG. 5B) as depicted in the cross-sectional view of FIG. 5B. In such embodiments, a cerclage material such as a cerclage wire is routed through passageway 532 (FIG. 5B) by inserting the cerclage material into aperture 524a through passageway 532 (FIG. 5B), whereupon the cerclage material exits passageway 532 (FIG. 5B) at aperture 524b. Such method of routing the cerclage material allows the tapered configuration of protruding mating face 528a to guide the cerclage material to a centrally-located aperture 526a to maximize the potential that the cerclage material is fed directly into centrally-located aperture 526b of receiving mating face 528b. In some embodiments of the present invention such as that depicted in FIG. 5B, the aperture located in the receiving face may have a larger diameter than the aperture located in the protruding face to facilitate passage of the cerclage material from cannulated tip 520a into cannulated tip 520b. In alternate embodiments, the cerclage material may be routed in a reverse direction and the protruding face may have an aperture having a larger diameter than the corresponding aperture located in the receiving face. Although FIG. 5 depicts a cup and cone style mating face interlock, other interlock styles may be substituted without departing from the scope of the present invention.

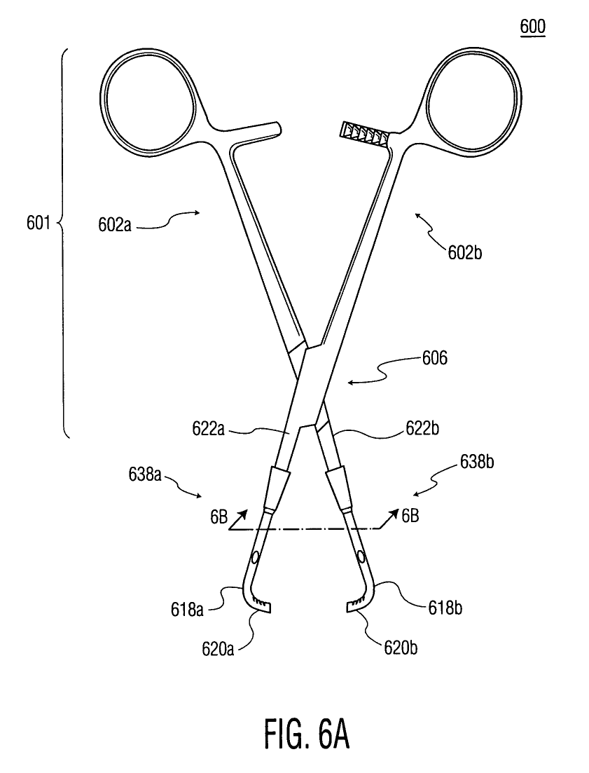

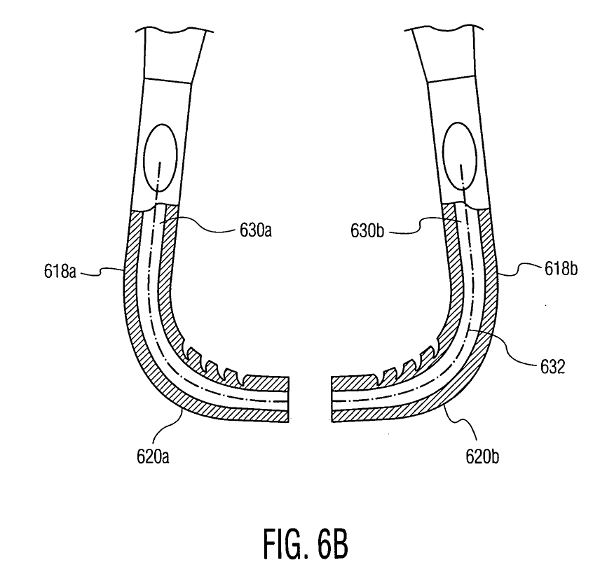

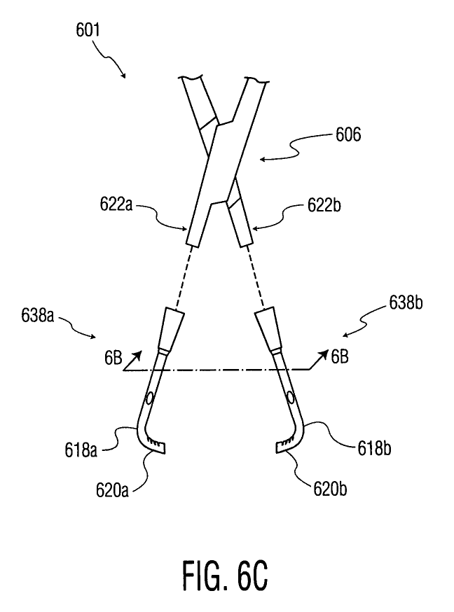

Referring now to FIG. 6A, depicted is implement 600 having detachable tips in accordance with one embodiment of the present invention. Implement base 601 includes, inter alia, handles 602 and hinge 606, which may have similar features to handles 102 and hinge 106 as discussed in greater detail above with respect to FIG. 1. However, jaw members 622 are detachably connected to detachable ends 638, which include cannulated members 618 and cannulated tips 620. Cannulated members 618 and cannulated tips 620 are similar to and have many of the features of cannulated ends 118 and cannulated tips 120, also as described in greater detail above with respect to FIG. 1. However, cannulated members 618 and cannulated tips 620 are integral to detachable ends 638 such that they may be discarded after use (e.g., to avoid and/or minimize sterilization of difficult to reach areas such as passageways 630 as depicted in FIG. 6B by allowing a user to discard the tips such as tips 620 after one or more uses and regularly sterilize only the implement base such as implement base 601) or may be removed to facilitate cleaning and/or sterilization. As depicted in FIG. 6B, passageways 630 and 632 are similar to passageways 330 and 332, respectively, as described in greater detail above with respect to FIGS. 3A and 3B.

Turning next to FIG. 6C, depicted is an exploded view of implement 600 as depicted in FIG. 6A. In one embodiment of the present invention, the proximal ends of detachable ends 638 include sleeves 640, which are configured to slide over jaw members 622. For example, sleeves 640 may have an inner diameter sized to form a compressive fit with the outer diameter of jaw members 622 when sleeves 640 are slid thereupon. However, other means of securing sleeves 640 to jaw members 622 may be substituted without departing from the scope of the present invention. Additionally, mechanisms and/or methods other than sleeves and/or compressive fits may be substituted for securing detachable ends 638 to jaw members 622 without departing from the scope of the present invention. For example, detachable ends 638 may be locked or snapped onto implement base 601 to minimize the possibility that such ends will accidentally disassociate with the implement base, potentially falling into the fracture site. Such locks or snaps may be configured to lock or snap into place upon a first depression by the user and to unlock or unsnap (from the implement base) upon a second depression by the user. However, the present invention is not so limited.

In another aspect of the present invention, jaw members of the implement base and/or detachable ends such as detachable ends 638 include a lock or other mechanism that requires such ends to be affixed to the implement base in a specific, predetermined configuration relative to the implement base. Such mechanism increases the likelihood that the mating faces of the tips of the detachable ends mate when the implement is indexed to a closed position. For example, the distal portions of the jaw members of the implement base (relative to the hinge or the like of the implement) may be manufactured with male or female components (e.g., a component approximating the head of a Phillips screwdriver) designed to mate with corresponding female or male components, respectively, located at the proximal portions of the detachable ends. However, other means of ensuring mating of the tips of the detachable ends may be substituted without departing from the scope of the present invention.

In other aspects of the present invention having detachable tips, such tips may be produced in a variety of sizes and/or configurations to allow the user to select the most suitable pair of tips for the surgery to be performed. Similarly, such tips may include passageways having varying sizes and/or configurations. For example, each tip may be specifically manufactured for application of cerclage to a specific bone. Or, each tip may be specifically manufactured for a specific method of applying cerclage. In addition, the varying tips may be sized to fit differing jaw members 622 or they may be sized to fit a single set of jaw members such that each pair of detachable tips may be used with the same implement base. The implements and/or tips may be distributed in a set to accommodate a variety of applications.

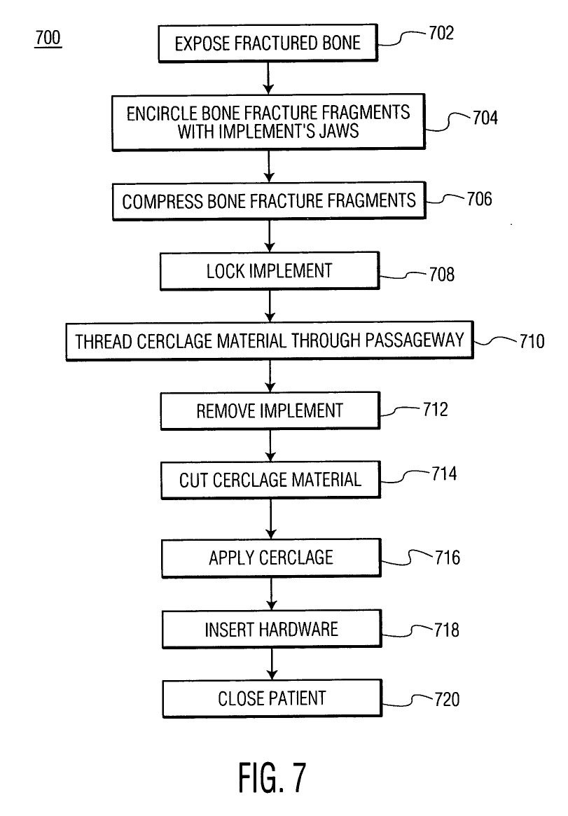

Referring now to FIG. 7, depicted is an exemplary method for applying cerclage to bone fracture fragments in accordance with one embodiment of the present invention. Process 700 begins at 702, at which the operative field is prepared and the fractured bone is exposed such that surgery may be performed. For example, the implement operator may start with an incision in the area of the fracture, as is known in the art. Following incision, the skin and muscles are retracted such that they do not obstruct the operative field, thereby providing access to the fractured bone. For example, for a metacarpal fracture, a dorsal hand incision may be made followed by displacement and retraction of the overlying extensor tendon. Once the extensor tendon is retracted, the operator has access to the metacarpal fracture site. Process 700 then proceeds to step 704.

After the fractured bone is exposed, the operator inserts the jaws of an implement of the present invention, such as implement 100 as discussed above with respect to FIG. 1, around the bone fracture fragments at step 704. In some embodiments of the present invention, the operator selects the appropriately sized implement from a set of implements, wherein each of the implements in the set has a varying distance or other area between its jaw, as measured when the jaws are in a closed position. That is, an implement may be selected from the set such that the distance or other area between its jaws is of sufficient size to completely encircle the bone fracture fragments while allowing the tips of the jaw members to make complete contact with each other such that a cerclage material or the like may be passed therethrough. Furthermore, an implement should be selected having a small enough distance or other area between its jaws to allow the clamp to hold and/or compress the bone fracture fragments in place as cerclage is performed. That is, although an implement may have a large enough distance or area between its jaws to completely encircle the area to which cerclage shall be applied, such implement may not be suitable if it does not allow the jaws to hold the bone fracture fragments in place as cerclage is performed. In the metacarpal fracture example, implement 100 is sized for metacarpal surgery such that jaws 110 fit securely around the metacarpal shaft. In some embodiments of the present invention, each implement in the set is designed and/or sized to accommodate a specific cerclage application. However, methods of the present invention are envisioned in which a single implement, rather than a set of implements, are used.

In embodiments of the present invention in which an implement such as implement 100 is used, the implement jaws are typically placed circumferentially around the fracture area. In some aspects of the present invention, the jaws of the implement may be inserted by placing them against the fractured area in a semi-closed position and gradually opening the implement's handles such that the jaws push surrounding soft tissue out of the way. For example, when applying cerclage to an exemplary metacarpal fracture, the operator inserts the implement around the metacarpal shaft by placing the implement against the fractured bone, gradually opening the implement and placing the jaws around the fracture site while avoiding the surrounding soft tissue. Once the jaws of the implement encircle the bone fracture fragments, process 700 continues to step 706.

At 706, the bone fracture fragments are compressed to reduce the fracture. In some aspects of the present invention, the implement operator manipulates the jaws of an implement of the present invention such as jaws 110 to reposition each of the bone fragment fractures back to its original or its desired position. The clamping action of the jaws aids the implement operator in drawing the bone fracture fragments together such that cerclage may be applied. Process 700 then proceeds to step 708.

At 708, once the bone fragment fractures are clamped in the proper position and, therefore, the implement is in its closed position, the jaws of the implement may be locked in place via a locking mechanism such as locking mechanism 112 as discussed in greater detail above with respect to FIG. 1. Locking of the implement retains the jaws in a closed position such that the mating faces of the cannulated tips of such jaws physically contact and form a continuous pathway for applying cerclage. Engagement of the implement's locking mechanism also frees the implement operator's hands to perform other tasks while maintaining compression of the fracture.

An illustration of an implement 800 in a locked position such that its jaws 810 encircle metacarpal bone 842 is depicted in FIG. 8. As depicted, jaws 810 of the implement apply pressure to the metacarpal bone while cannulated ends 818 and tips 820 (similar to cannulated ends and tips 118 and 120, respectively as discussed above with respect to FIG. 1) of jaws 810 encircle the bone. Cannulated ends 818 and tips 820, respectively, provide a contained passageway for the cerclage material that retains such material within the passageway such that it does not damage or disrupt surrounding tissue, skin, and the like during threading of the material around the fracture site.

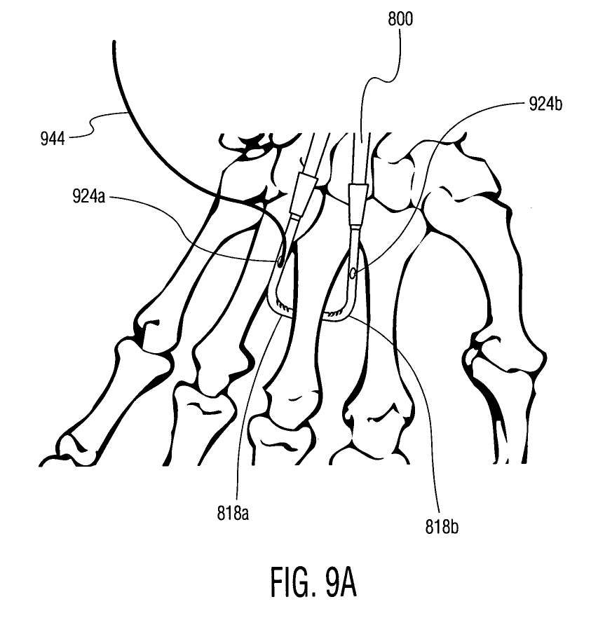

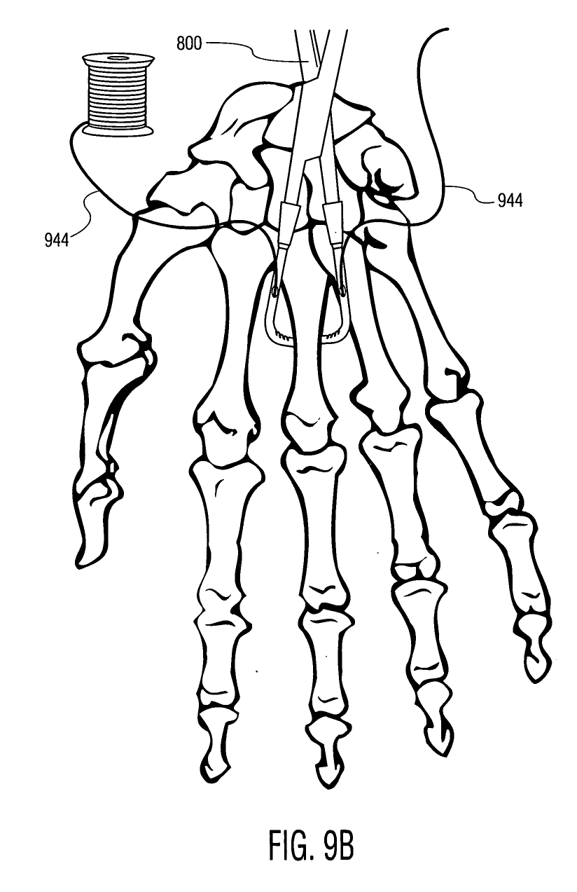

Referring back to FIG. 7, once the implement has been locked, process 700 proceeds to 710. At 710, a cerclage material is threaded through the continuous pathway formed during step 708. The enclosed path allows easy passage of the cerclage material without interference from the surrounding soft tissue. An illustration of such threading is depicted in FIGS. 9A and 9B. As depicted in FIG. 9A, the implement operator feeds a first end of cerclage material 944 into a first aperture 924a of implement 800. Thereafter, the implement operator continuously feeds cerclage material 944 through aperture 924a, thereby forcing the first end of cerclage material 944 through cannulated ends 818 and cannulated tips 820 (not shown in FIG. 9A) until such end exits the opposing aperture 924b. Once the first end of cerclage material 944 exists opposing aperture 924b, the implement operator may continue to feed cerclage material 944 through implement 800, or the implement operator may grasp the first end with a hand or a second implement (e.g., surgical clamp, surgical clasp, etc.), as known in the art, and pull cerclage material 944 through implement 800 until the desired length of cerclage material 944 extends beyond aperture 924b. FIG. 9B depicts implement 800 and cerclage material 944 after the conclusion of step 710 in accordance with one embodiment of the present invention.

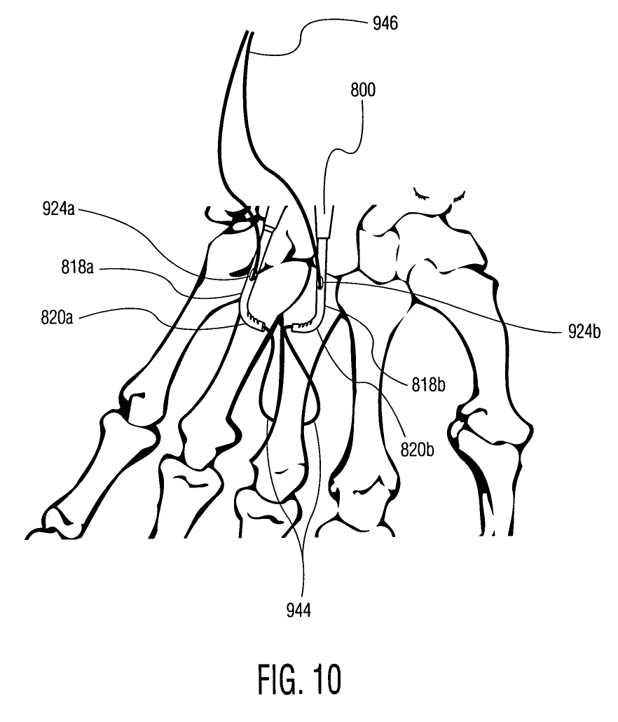

Turning back to FIG. 7, after the cerclage material has been threaded through the implement, process 700 proceeds to step 712 at which the implement is removed. Such removal may be performed using any one of a variety of methods. In one embodiment of the present invention, the implement is removed by opening its jaws as necessary to allow them to clear the fracture site upon removal. Next, the jaws are pulled from the fracture site while the cerclage material remains encircled about the bone fracture fragments. Such removal is illustrated in greater detail in FIG. 10 with respect to removal of implement 800 from the exemplary metacarpal bone fracture site. As illustrated, cerclage material 944 is pulled through implement 800 as it is removed from the site. In one aspect of the present invention, the implement operator threads a sufficient length of cerclage material 944 through implement 800 in step 710 to allow the implement to retain cerclage material 944 within cannulated ends 818 throughout removal of implement 800. That is, the length of cerclage material 944 threaded through implement 800 should be such that first end 946 of cerclage material is not pulled through aperture 924b and cannulated end 818b during removal of implement 800. Such pulling through may result in cerclage material 944 falling into the bone fracture site, which will increase the difficulty and time required to apply cerclage to the bone, as this will require careful removal of cerclage material 944 from the bone fracture site without imparting damage thereto. Although the specific aforementioned process of removing implement 800 has been detailed, other methods may be substituted without departing from the scope of the present invention.

Referring back to FIG. 7, at step 714, the implement operator cuts the cerclage material. After implement 800 has been removed to expose the desired length of cerclage material 944 as depicted in FIG. 10, the implement operator grasps the portions of cerclage material 944 adjacent to cannulated tips 820 to prevent such material from falling into the bone fracture site. Thereafter the cerclage material adjacent to cannulated tip 820a is cut or otherwise severed such that implement 800 may be completely removed. During such removal, first end 946 of cerclage material is pulled through aperture 924b and cannulated end 818b such that implement 800 is completely removed and the implement operator retains first end 946 and the severed end of cerclage material 944 in his or her hand. By maintaining contact with the fracture site as implement 800 is gradually opened and removed, the possibility that soft tissue will reenter the site is reduced. Also, after implement 800 is removed, cerclage material 944 remains in the proper position for application of cerclage. Although the specific aforementioned process of cutting cerclage material 944 has been detailed, other methods may be substituted without departing from the scope of the present invention. For example, the implement operator may rotate the implement multiple times to affix the two leads of the cerclage material together prior to severing such material. In some aspects of the present invention, such rotation may be used to apply cerclage to the fracture prior to cutting cerclage material 944 and/or removal of implement 800. Process 700 then proceeds to 716.

At 716, the operator applies cerclage to the fracture. Such application may be achieved by manually tightening the cerclage material and cinching, tying, twisting, or otherwise securing the cerclage material to retain it in place using methods commonly known in the art. Also as known, care must be taken to avoid over tightening the cerclage material as such over tightening may result in material breakage. Process 700 then optionally proceeds to step 718.

At 718, hardware such as rigid plates, screws, or the like may be optionally installed. Step 718 is optional as application of cerclage alone will be sufficient for repair of some fractures. However, in other cases, further internal fixation may be required. In either case, application of cerclage to a fracture achieves maintenance of reduction and bony fixation, which can be supplemented by more rigid forms of fixation with interfragmental screws, dynamic compression plates, Kirshner wires, and/or application of the tension band principle. Furthermore, the preliminary application of cerclage in accordance with the present invention enables a single operator to quickly reduce a fracture and apply any additional hardware (e.g., rigid fixation means) without additional assistance. Also, the preliminary application of cerclage in accordance with the present invention allows additional hardware to be more easily placed without losing the reduction achieved with the implement, thereby reducing operating time.

Process 700 proceeds to step 720, at which the exposed fracture site is closed via replacement of the surrounding tissue and/or skin and securing same via stitching or other methods as known in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An apparatus for applying cerclage to a plurality of segments comprising:
two jaw members, each of said jaw members including at least one cannulated end, and each of said cannulated ends including at least two apertures, and at least one of said apertures is located at each terminus of said cannulated end;
two handle members for opening and closing said jaw members relative to each other; and
at least one hinge for coupling said jaws to each other and for coupling said jaw members to said handle members;
wherein said jaw members are configured relative to each other to draw said plurality of segments together to a desired position as an operator manipulates said jaw members from an open position to a closed position; and
wherein said cannulated ends are configured relative to said jaw members to engage each other when said apparatus is manipulated to a closed position; and
wherein said engagement mates cannulated portions of each of said cannulated ends together to form one continuous passageway through which a cerclage material may be threaded.

2. An apparatus according to claim 1,
wherein said jaw members are pivotally coupled to each other; and
wherein such pivoting rotates said jaw members from said open position to said closed position.

3. An apparatus according to claim 1 further comprising:
at least one locking mechanism for locking said jaw members in said closed position.

4. An apparatus according to claim 3, wherein said locking mechanisms includes a pair of toothed protrusions having interlocking teeth.

5. An apparatus according to claim 1, wherein at least a portion of said cannulated end is approximately aligned with a longitudinal axis of at least one of said handle members.

6. An apparatus according to claim 1, wherein said plurality of said segments are bone fracture fragments.

7. An apparatus according to claim 1, wherein inwardly facing surfaces of said continuous passageway are configured to facilitate unimpeded movement of said cerclage material therethrough.

8. An apparatus according to claim 1, wherein at least one of the group consisting of said jaw member, said cannulated member, and combinations thereof include at least one serration for performing at least one of the group consisting of aiding said drawing together of said plurality of said segments, increasing a grasp of said segments by said serrations, increasing a friction between said serration and said segments, and combinations thereof.

9. An apparatus according to claim 1, wherein at least one of the group consisting of said jaw members, said hinge, said handle members, and combinations thereof is sized based upon at least one of the group consisting of a size, a type, and combinations thereof of said plurality of said segments to which said cerclage shall be applied.

10. An apparatus according to claim 1, wherein said cannulated end includes at least one cannulated tip, each cannulated tip includes at least one mating face, and each mating face includes at least one aperture.

11. An apparatus according to claim 10, wherein at least one of the group consisting of a first of said cannulated tips, said mating face of said first of said cannulated tips, and combinations thereof is configured to interlock with at least one of the group consisting of a second of said mating tips, said mating face of said second of said cannulated tips, and combinations thereof.

12. An apparatus according to claim 11,
wherein at least one of the group consisting of a first of said cannulated tips, said mating face of said first of said cannulated tips, and combinations thereof is configured as a cone; and
wherein at least one of the group consisting of a second of said mating tips, said mating face of said second of said cannulated tips, and combinations thereof is configured as a cup.

13. An apparatus according to claim 10, wherein said aperture of a first of said cannulated tips has a larger diameter than said aperture of a second of said cannulated tips.

14. An apparatus according to claim 10, wherein said cannulated tip is configured to guide said cerclage material to said aperture of said cannulated tip.

15. An apparatus according to claim 10, wherein said cannulated tip is tapered.

16. An apparatus according to claim 1 further comprising:
at least one identification device.

17. An apparatus according to claim 1, wherein said handle members, said hinge, and a portion of said jaw members are configured as at least one of the group consisting of a surgical clamp, a surgical clasp, and combinations thereof.

18. An apparatus according to claim 1,
wherein a longitudinal axis of a first portion of said cannulated end is downwardly angled relative to a longitudinal axis of at least one of said jaw members; and
wherein a longitudinal axis of a second portion of said cannulated end is approximately perpendicular to a longitudinal axis of at least one of said jaw members.

19. An apparatus according to claim 1, wherein said cannulated end is detachable from said jaw member.

20. A method for applying cerclage to a plurality of segments comprising the steps of:
providing a cerclage implement having jaw members, said jaw members pivotally coupled to each other, and each of said jaw members including at least one cannulated end;
surrounding said plurality of said segments with said jaw members;
drawing said plurality of said segments together to a desired position via pivoting of said jaw members while simultaneously engaging said cannulated ends of said jaw members, said engagement mating cannulated portions of each of said cannulated ends together to form one continuous passageway through which a cerclage material may be threaded;
threading at least one cerclage material through said continuous passageway;
removing said cerclage implement from said plurality of segments; and
securing said cerclage material.

21. A method according to claim 20, wherein pivoting said jaw members relative to each other varies a distance between inwardly facing surfaces of said jaw members.

22. A method according to claim 20 further comprising:
locking said cannulated ends in an engaged position.

23. A method according to claim 20, wherein said plurality of said segments are bone fracture fragments.

24. A method according to claim 20,
wherein said cerclage instruments are provided in a plurality of sizes; and
wherein said size of said cerclage instrument is selected based upon at least one of the group consisting of a size, a type, and combinations thereof of said plurality of said segments to which said cerclage shall be applied.

25. A method according to claim 20 further comprising:
attaching said cannulated end to said cerclage instrument prior to said surrounding said plurality of said segments with said jaw members.

26. A method according to claim 25, wherein said cannulated ends are selected based upon at least one of the group consisting of a size, a type, and combinations thereof of said plurality of said segments to which said cerclage shall be applied.

27. A method according to claim 20 further comprising:
removal of said cannulated ends from said cerclage instrument after removal of said cerclage instrument from said plurality of said segments.

28. A method according to claim 20 further comprising:
pushing material surrounding said plurality of said segments away from said segments via pivoting of said jaw members.

29. A method according to claim 20 further comprising:
severing said cerclage material to aid in at least one of the group consisting of said removal of said cerclage instrument from said plurality of said segments, said securing of said cerclage material, and combinations thereof.

30. A method according to claim 20 further comprising:
affixing additional hardware to said plurality of said segments.
